(19) United States
(12) Patent Application Publication
(10) Pub. No.: US 2008/0318801 A1
(43) Pub. Date: Dec. 25, 2008

(54) METHOD AND KIT FOR EVALUATING RNA QUALITY

(76) Inventor: Conrad L. Leung, Edison, NJ (US)

Correspondence Address: BAKER BOTTS L.L.P., 30 ROCKEFELLER PLAZA, 44TH FLOOR, NEW YORK, NY, 10112-4498, US

(21) Appl. No.: 12/048,939

(22) Filed: Mar. 14, 2008

Related U.S. Application Data
(63) Continuation of application No. PCT/US06/42160, filed on Oct. 26, 2006.
(60) Provisional application No. 60/731,563, filed on Oct. 28, 2005.

(51) Int. Cl. C40B 30/04; C12Q 1/68

(52) U.S. Cl. 506/9; 435/6

(57) ABSTRACT

The present invention relates to a method for analyzing and rapidly determining the quality of a test RNA of unknown quality utilizing a novel quantitative reverse transcription-polymerase chain reaction (RT-PCR) based method. The present invention is based on normalizing the 3′ end of the house-keeping gene glyceraldehyde-3-phosphate dehydrogenase (GAPDH) to the relative abundance of the 5′ end of GAPDH MRNA in the test RNA. The present invention is particularly useful in pre-screening postmortem tissue samples for microarray experiments and for evaluating large quantities of samples which would be time consuming and expensive to analyze by methods currently in use.

### CROSS-REFERENCE TO RELATED APPLICATION

This application claims priority to U.S. Provisional Application No. 60/731,563 filed Oct. 28, 2005, the contents of which is incorporated herein in its entirety.

### 1. INTRODUCTION

The present invention relates to a novel quantitative reverse transcription polymerase chain reaction-based method to evaluate RNA quality. The method is particularly useful in pre-screening postmortem tissue samples for microarray experiments.

### 2. BACKGROUND OF THE INVENTION

### 2.1 Microarray Analysis

A recently developed technology variously referred to as gene expression profiling, high-throughput expression analysis, GeneChip® technology, biochips and microarray analysis, is finding increasing utility in many areas of biomedical research. This technology makes use of miniaturized, high-density arrays of DNA or oligonucleotide probes in various formats to assess specific genetic information of interest in a given biological context (Watson et al. 1998, Curr Opin Biotechnol. 9(6):609-14; Buesa et al. 2004, J Neuropathol Exp Neurol. 63(10):1003-1014).

Typically, a microarray is comprised of an array of nano-spotted immobilized nucleic acids on an inert substratum in which each spot is of known sequence identity corresponding to a specific cellular gene. An individual array usually comprises several thousand spots corresponding to a specified classification, such as genes expressed in a given species, cell type, tissue etc. The technique is performed by hybridizing a suitably labeled sample of nucleic acid from a biological source to the immobilized microarray. Positive signals resulting from specific hybridization of the labeled sample to the immobilized genes on the microarray are detected using a detection device. The data obtained from such analyses provides information about the identity and potential role of specific genes involved in the phenomenon under study. As opposed to traditional gene detection and expression level quantitation techniques such as dot-blot or Northern blot analysis, the results of microarray-based studies are accessed through specialized detection devices or readers. The signal intensity generated by hybridization is recorded at each spot of the array, enabling the precise identification as well as the simultaneous quantitation of the corresponding RNA transcripts being expressed. Thus microarray based studies, in most instances, require the utilization of dedicated and specialized instruments and equipment.

It is of particular note that microarray technology permits virtually simultaneous detection and quantitation of several thousand sequences, previously impossible using conventional methods. This technology thus provides a powerful tool to study global gene expression patterns on a scale unimaginable and technically impossible prior to the invention of this technology. Data obtained from microarray analyses may be used to identify which genes are expressed specifically in a particular cell, tissue, disease state or following a particular treatment thus enabling the evaluation of the role of one or a group of genes in each particular context. This technology has been successfully utilized to investigate genes associated with a particular type of cancer, to determine global interaction profiles of genes, and to perform genetic screening and diagnosis in certain disease conditions (Alizadeh et al, 2001, J. Pathol. 6:195(1):41-52; Espina et al 2005, Cancer Invest. 23(1):36-46; Pahl 2005, Expert Rev Mol Diagn. 5(1):43-52).

### 2.2 Cellular RNA Species and Microarray Analyses

The phenotype of a specific eukaryotic cell type or tissue is initiated and maintained, at least in part, by the differential transcription of unique subsets of DNA gene sequences. The products of transcription may function directly in the form of ribosomal-, transfer- or other small RNA species having catalytic or regulatory functions. A quantitative minority of total cellular RNA is so-called messenger- or mRNAs, which are translated into proteins.

Non-protein encoding RNAs play an important and indispensable role in cellular maintenance, growth and survival. However, protein encoding genes and their corresponding mRNAs are generally of greater interest to researchers. The vast majority of microarray analyses of gene expression are based on determining the on/off state and comparative level of expression of a large number of protein encoding genes from a source of interest as compared to a relevant control sample. The starting material for microarray analysis comprises isolated and purified total RNA or mRNA from a source of interest. The occurrence of expression (the on/off state) of a given gene in a sample is measured by a positive or negative signal on the microarray. The relative abundance of a given gene is in turn determined by the intensity of the hybridization signal obtained during microarray analyses. Thus, importantly, both gene activity and the relative level of gene expression may be measured by performing microarray analyses (Iyer et al. 1999, Science 283:83-87; Alizadeh et al, 2001, J. Pathol. 195(1):41-52; Espina et al 2005, Cancer Invest. 23(1):36-46; Pahl 2005, Expert Rev Mol Diagn. 5(1):43-52).

### 2.3 Factors Affecting Microarray Analyses

Many factors affect the final result and the quality of a microarray analysis. Some of these include the type of array utilized including the subset of genes contained on the array; the length of arrayed probe nucleic acid; the algorithms utilized in data analysis etc. In addition, study of gene expression by microarray analysis requires the isolation of RNA from the cells or tissue samples being studied. The isolated RNA used to make the labeled probe provides a snapshot of the expression profile of all genes being expressed in that given biological sample at the time of isolation. The quality of RNA utilized to generate microarray probes is therefore a critical factor in the success of a microarray based experiment (Schoor et al. 2003, Biotechniques 35(6):1192-1201).

A major factor affecting microarray analyses is the relative instability of RNA molecules in general, due to the ubiquity and high stability of RNA degrading enzymes, the RNases. Individual mRNA species vary widely in their relative abundance and stability within cells and many of the less abundant mRNAs may be easily destroyed during the process of RNA isolation from a biological source (Schoor et al. 2003, Biotechniques 35(6):1192-1201; Miller et al. 2004, Biotechniques 36(4):628-633). A consequence of mRNA instability and loss of less abundant species is the likelihood of arriving at erroneous conclusions based on studies derived from non-representative partially degraded samples of test material. Consequently, the quality of information derived from many forms of microarray analyses and the reliability of conclusions obtained from such analyses is critically dependent on the quality of RNA used to probe the microarray (Schoor et al. 2003, Biotechniques 35 (6):1192-1201).

### 2.4 Microarray Analysis of Human Tissues

Conventional pathological analysis of human tissue samples is prohibitively expensive and time consuming. Human tissue samples are amenable to microarray analyses and this alternative is potentially less costly on a per sample basis than conventional pathological methods. Microarray analyses of RNA samples derived from small portions of a human tissue sample enable the utilization of extremely small amounts of tissue and permit the comparative analysis of a large number of tissue samples.

Microarray technology has the potential to greatly facilitate the translation of basic research into clinical practice. Potential applications include the establishment of associations between molecular changes and clinical endpoints, testing of potential therapeutic targets using tissue samples from specific cancer patients, standardization of molecular detection of targets, and rapid translation of results from cell lines and animal models to human cancer. Thus microarray analysis of human tissue samples has the potential to increase the scope of molecular diagnostics and pharmacogenomics. Microarray analysis is already currently utilized in molecular disease classification and drug development (Hollingshead et al. 2005, Neurobiology of Disease 18:649-655). In addition, the clinical importance of a candidate gene identified in a screening procedure may be validated by microarray analysis of human tissues derived from a relatively large number of individuals on a larger scale than would be feasible by other methods.

In general, biological material is exposed to a wide spectrum of collection, handling and storage variables. For example, a surgical biopsy or necropsy specimen may have been subjected to ambient temperature, prior to refrigeration, for a period of time sufficient to allow selective RNA degradation. Therefore, when a tissue sample is subjected to microarray analyses the issue of quality of the sample becomes very important. The quality of a sample relevant to microarray analysis is the quality of the RNA it contains. Thus a sample of good quality should have relatively undegraded RNA representative of the original state of the sample before isolation, processing and storage. Valid comparisons by microarray analysis may only be made between samples of similar type and quality. A sample must therefore be assessed to determine whether it meets the quality standard for microarray analysis to avoid waste of resources and ensure that valid results are generated.

When embarking on a microarray based study of a particular phenomenon statistical validity is usually of critical importance. A study will typically require the analysis of samples collected from multiple sources and utilizing differing handling methods. To ensure that analysis is being performed on samples of comparable quality, pre-testing each sample is usually recommended. A test microarray of samples to be subjected to the actual analysis may be performed to determine the quality of RNA in each. The utilization of microarray analyses to merely determine the quality of a test RNA sample has potential drawbacks. The cost of performing such an analysis is not cost-effective. Multiple steps are involved including sample preparation, labeling, hybridization, and analysis, all of which add to costs and turn-around time. If the resulting microarray analysis merely determines that the tested RNAs are of poor quality, considerable resources would have been expended without any corresponding return. A quicker and cheaper non-microarray based, but equally reliable method to determine the suitability of utilizing a given test RNA sample for microarray analysis will be very useful.

The invention described herein provides for an inexpensive and efficient means for determining the quality of a test RNA sample. It may be utilized in pre-screening biopsy or postmortem tissue samples to determine if RNA derived from such samples are suitable material to perform microarray studies.

### 3. SUMMARY OF THE INVENTION

The present invention relates to a method for analyzing and rapidly determining the quality of a test RNA utilizing a novel quantitative reverse transcription polymerase chain reaction (RT-PCR)-based method. The method comprises: (i) determining the relative normalized amount of 5′-end of a target mRNA in a test RNA of unknown quality and a standard RNA of known, good quality; (ii) determining the numerical ratio of 5′-end abundance in standard to test RNA; and (iii) rating the relative quality of one or more unknown test RNAs based on the numerical value obtained for each sample of test RNA where a smaller ratio indicates that the RNA is of better quality.

RNases that typically degrade mRNA act by binding to and cleaving ribonucleotide bonds. Cleavage of a given RNA strand may either be at an internal site if the RNase is an endoribonuclease or at the 3′ or 5′-end if the enzyme is an exoribonuclease. Cellular RNases are generally a combination of both types of enzymes in varying proportion depending on the cell or tissue type. When good quality mRNA is reverse transcribed using an oligo-dT primer (an oligonucleotide containing a stretch of generally 18- to 24 T-residues) to prime cDNA synthesis from the 3′ poly-A tail, a larger amount of full length cDNA is generated compared to that in a similar reaction using test RNA samples with relatively degraded and fragmented mRNA. Therefore, the ratio of 3′-end specific PCR product to 5′-end specific PCR product in a good quality test mRNA sample should have a value of close to or equal to 1 (FIG. 1).

Determinations of mRNA quality based on the 5′-end to 3′-end ratio of a target mRNA utilizing the current method of choice, namely the Genechip® Test3 array (Affymetrix™) were found to correlate with the results obtained by the present invention. The present invention provides for testing the quality of a test RNA from any biological source and is particularly useful in pre-screening RNA isolated from biopsy or postmortem tissue samples for microarray experiments and for evaluating large quantities of samples which would be time consuming and expensive to analyze by existing methods including the Genechip® Test3 array.

### 4. BRIEF DESCRIPTION OF THE FIGURES

FIG. 1. Analysis of RNA quality by a quantitative RT-PCR based method: A schematic representation of the strategy used to monitor the quality of test RNA samples is shown. RNA Sample 1 and RNA Sample 2 have 2-fold and 4-fold lesser relative abundance of full length mRNA, respectively, of intact target GAPDH mRNA. Therefore test RNA Sample 1 and RNA Sample 2 also have 2- and 4-fold lower amounts of amplifiable GAPDH mRNA 5′ ends compared to the standard. The “5′-end fold difference” is the ratio of: [the abundance of the 5′-end product in the standard sample] to [the abundance of the 5′-end product in the test sample]. As seen in the last line of the figure a better quality test RNA exhibits a smaller numerical value 5′-end fold difference compared to a poorer quality test RNA. The small horizontal arrows represent PCR primers which are positioned to indicate the region of cDNA amplified. The stretch of A residues at the right-hand ends of target GAPDH mRNAs represent, poly-A tails used to prime cDNA synthesis using an oligo-dT primer as shown.

FIG. 2. Comparison of the 3′ to 5′ ratio of target GAPDH as determined by Genechip® and its 5′ relative abundance as determined by quantitative RT-PCR: A: PCR primer sequences of the GAPDH gene: The primers schematically represented in FIG. 1, utilized in the PCR reactions plotted in FIG. 2B are shown including, GAPDH-5° F. (SEQ ID NO:1), GAPDH-5′R (SEQ ID NO:2), GAPDH-3° F. (SEQ ID NO:3) and GAPDH-3′R (SEQ ID NO:4). B: The relationship of GAPDH 3′ and 5′ end ratio by two alternative methods: A scatter plot showing the relationship between the 3′- to 5′-end ratio of GAPDH determined by Genechip® (Y-axis) and the fold difference of GAPDH 5′ ends determined by quantitative RT-PCR (X-axis). Each data point represents the value obtained with a given brain RNA sample. The result obtained by Genechip® analysis or by the present invention may be read off the corresponding numerical scale on the Y-axis or X-axis respectively for that individual point. The data point at coordinate (0, 1) is from a human cell line that showed no RNA degradation. A linear regression fit is plotted as a line with the equation shown. The coefficient of determination (R²) corresponds to the square of the correlation coefficient.

### 5. DETAILED DESCRIPTION OF THE INVENTION

The present invention provides for a method to determine the quality of a test RNA, and for kits that may be used to practice the method. The term “quality” as used herein, is a measure, expressed as an arbitrary numerical value, of the proportion of undegraded or unhydrolyzed mRNA molecules in the sample. The arbitrary numerical value based on the 2^{-ΔΔC}_{T} method (Livak and Schmittgen 2001, Methods 25:402-408) enables comparison of distinct and independently prepared RNAs of unknown quality. This numerical value is derived from a ratio of undegraded or unhydrolyzed mRNA molecules in a test RNA compared to undegraded or unhydrolyzed mRNA molecules in a standard RNA, wherein the mRNA in the standard is known to have minimal mRNA degradation. An undegraded or unhydrolyzed mRNA molecule is one in which the protein coding region or so-called open reading frame and associated flanking 5′ and 3′ untranslated regions or UTRs are present in a contiguous unbroken or uninterrupted single strand of RNA arranged in a linear 5′ and 3′ orientation originally encoded by chromosomal or other genomic DNA. The sequence of a complete undegraded or unhydrolyzed mRNA may be determined, if not previously known, by standard sequencing techniques, or if known, by utilizing a previously reported cDNA sequence in a publication or DNA sequence database.

The method of the invention comprises the following steps:
(i) isolation and purification of total and/or polyA⁺ from a test RNA;
(ii) synthesis of cDNA from the said test RNA;
(iii) performing quantitative or real time PCR reactions on a cDNA utilizing a primer pair to quantitate a region in the 5-end of a target mRNA and a primer pair to quantitate a region in the 3′-end of the same target mRNA in said test RNA;
(iv) calculating the relative abundance of the 5′-end of the target mRNA in the test RNA population by using the 2^{-ΔΔCt} method; and
(v) determining the quality of the test RNA by calculating the 5′-end fold difference of a target mRNA in the test RNA when compared to the standard RNA.
For clarity and not by way of limitation, the detailed description of the invention is divided into subsections describing each of these steps.

### 5.1 Overview

The present invention relates to protein coding messenger- or mRNA which make up between 1-5% of total cellular RNA when compared to the average amount of other types of RNA present in eukaryotic cells including, transfer-RNAs, ribosomal-RNAs and small nuclear RNAs. RNA molecules are recognized to have a specific 5′ to 3′ orientation and thereby also posses defined 5′ and 3′ termini. Most mRNAs contain a so-called “poly-A tail” at the 3′ end comprising of between 50 to 250 adenylate residues and a 5′-end conserved motif called the “Cap” (Baralle 1983, Int Rev Cytol. 81:71-106). The present invention relates to the known unstable and degradation-prone nature of RNA and in particular mRNA molecules due to the presence of RNA-degrading enzymes called RNases (Levy 1975, Life Sci. 17(3):311-6). RNA molecules are also known to hydrolyze in an alkaline environment and may be degraded when treated or stored under alkaline conditions. The present invention provides a means for pre-determining the quality of RNA prior to utilizing the sample in a microarray analysis, preparation of a CDNA library or other related analytical study. The present invention relates to the knowledge that use of a degraded RNA sample could generate erroneous results, misleading interpretations or result in a loss of resources and time. In one embodiment, the present invention relates to the analysis of multiple samples to determine the RNA quality of each with the understanding that performing such multiple sample analyses may be expensive, time consuming or inaccurate when performed with currently used methods.

### 5.2 Step (i): Isolation of RNA

### 5.2.1 Test RNA

The present invention provides for a test RNA, the quality of which is to be determined utilizing the present invention. The present invention encompasses isolation of test RNA from various sources. Thus the source of test RNA may include a eukaryotic, non-human animal or human cell or cells. Non-limiting examples include pure or mixed cultures of primary cells derived from non-human animal or human tissues. Other non-limiting examples include a homogenous population of cells or a mixed population comprising a “feeder-layer” of cells on top of which a distinct cell type such as a stem cell is grown in vitro. Further non-limiting examples include RNA derived from either normal immortalized cells or transformed cells derived from established tumor cell lines. In preferred embodiments, the source of RNA is one which is at risk for degradation of RNA such as a tissue biopsy or post-mortem tissue sample.

According to one non-limiting embodiment of the invention, a test RNA may be derived from tissue that has been subjected to laser capture or other microdissection procedures (Enunert-Buch et al. 1996, Science 274, 998-1001) to derive selected and specialized cell types relevant to an investigation or survey. In other non-limiting embodiments, a test RNA may be isolated from sources including but not limited to material derived from fresh tissue, frozen tissue, fixed tissue sections, cryostat sections, and paraffin embedded sections. The material may be a sample stored in an archive such as a tumor bank or tissue bank. In non-limiting examples a cell(s) may be collected from the body fluid of a human or non-human animal including but not limited to blood, saliva, semen, urine or other source.

In non-limiting embodiments, the source of test RNA may be a non-malignant cell arising in the brain, spinal cord, skin, throat, tongue, lung, stomach, intestine, liver, pancreas, kidney, breast, ovary, testicle, prostate, bone, muscle, lymphocyte, erythroblast, macrophage, dendritic cell etc. Such cell, while not malignant, may be normal or may be subject to a hereditary metabolic, degenerative or infectious disorder.

In other non-limiting embodiments, the source of test mRNA may be a malignant cell such as a glioblastoma cell, an astrocytoma cell, a medulloblastoma cell, a spinal cord cancer cell, a skin cancer cell, a thyroid cancer cell, a parotid gland cancer cell, a lung cancer cell, a stomach cancer cell, a colon cancer cell, a liver cancer cell, a pancreatic cancer cell, a renal cancer cell, a breast cancer cell, an ovarian cancer cell, a prostate cancer cell, a bone cancer cell, a connective tissue cancer cell, a leukemia cell, a lymphoma cell, a melanoma cell, etc.

### 5.2.2 The Standard RNA

The present invention provides for use of a standard RNA, wherein the standard RNA is of high quality and is not substantially degraded or hydrolyzed as described above. Preferably, the standard RNA has a 3′:5′ ratio, as determined by GeneChip® or RT-PCR as discussed herein, of at least about 0.6 to 1.5, preferably at least about 0.7 to 0.9 and most preferably 1.0. In a particular embodiment, the invention may utilize RNA from cells derived from in vitro tissue culture to make the standard RNA preparation. In a further non-limiting embodiment, the in vitro tissue culture cell may have specific properties particularly amenable or related to a study. Examples of such cells include but are not limited to cells known to have a particular lineage or origin such as cells from muscle, nervous tissue, liver, lung, heart, pancreas etc. such that utilization of the specific cell type enables an optimized use of the instant invention due to the specific property of the cell used. Thus for example, utilization of an astrocyte cell line from the brain may facilitate the analysis of the quality of RNA derived from brain tissue biopsies since rare brain-specific genes may be utilized to determine the quality of a test RNA sample. Thus in a specific non-limiting embodiment the instant invention encompasses the utilization of an in vitro cell line that matches a tissue type under analysis. In a further non-limiting embodiment the cell line used to make the standard RNA may be isolated from SW 13 cells, a human adrenal carcinoma cell line (ATCC Cat. No. CCL 105).

### 5.2.3 The Target RNA

The present invention provides for the evaluation of RNA quality by quantitation of relative amounts of the 5′-end region of a selected target mRNA from among the population of mRNAs in the test RNA. The 3′-end of the selected target mRNA is used to normalize the amount of 5′-end of a target mRNA between different test RNA samples (section 5.5). A target mRNA as provided for by the invention, may be virtually any mRNA molecule derived from a cellular gene. The target mRNA may or may not be expressed at comparative levels between the test RNA and standard RNA but should preferably be expressed in both populations. In one embodiment, the choice of the target mRNA may depend on the ultimate intended use of the test RNA to be tested to determine its quality i.e. a target RNA may be chosen for its known or desired properties. Such properties include but are not limited to high abundance and/or high mRNA stability, which are attributes accorded but not limited to the so-called cellular “house-keeping” genes. Specific non-limiting examples of such target mRNAs are GAPDH, β-Actin, cyclophilin etc. (Raff 1997, BioTechniques 23: 456; Haendler et al. 1990, Eur. J. Biochem. 190: 477; Spanakis et al. 1993, Nucl. Acids Res. 21: 3809; Mansur et al. 1993, Nucl. Acids Res. 21:993; Bhatia et al. 1994, Anal. Biochem. 216:223). Alternatively, the invention provides for a target mRNA chosen because of its low abundance and/or low stability. Specific non-limiting examples of such target mRNAs correspond to c-myc, c-fos, cyclin D1 etc. In a specific embodiment, a target mRNA may be chosen for its relatively small size so as to enhance the possibility of obtaining full length cDNAs or alternatively the choice of target mRNA may be a relatively large mRNA either to enhance the stringency of the quality determination or if the experimental system under study involves a population of large mRNA transcripts or alternatively if large sized mRNA are expected to make up the subject of a study. In one non-limiting example, the target mRNA may be selected to correspond to mRNA contained in a commercially available microarray. The invention also provides for a combination of more than one type of target RNA including but not limited to two distinct genes. If more than one target mRNA is used, these may have similar properties as set forth above (e.g. stable mRNAs with high abundance or unstable mRNAs with low abundance, large or small sized mRNAs etc.). Alternatively, the choice of multiple target RNAs may be based on a deliberate choice of mRNAs with entirely distinct abundance or stability properties as set forth above so as to determine the quality of test RNA with respect to high abundance and/or stability as well as unstable and low abundance mRNA species.

### 5.2.4 Methods for RNA Isolation

The present invention provides for methods for isolation of RNA to be used either as a test RNA or a standard RNA. Methods of RNA isolation and purification include but are not limited to the acid-phenol guanidine isothiocyanate procedure (Puissant and Houdebine. 1991, Biotechniques 8:148-149; Chomczynski and Sacchi, 1987, Anal Biochem. 162:156-159), the RNAzol procedure (Invitrogen, Carlsbad, Calif.) or CsCl banding procedure. In an alternate embodiment, methods permitting the simultaneous isolation of RNA and DNA, as opposed to RNA alone (e.g. the ToTALLY RNA™ isolation kit, Ambion Inc. Woodward, Tex.) is also included in the invention. In another non-limiting embodiment, the present invention provides for methods for the isolation of RNA from cell lines or tissue samples that may utilize RNA isolation kits including to but limited to those available from commercial suppliers. The invention provides for RNA isolation from commercially available kits irrespective of the specific method, chemistry or alternative forms of reagents used in the isolation. Non-limiting examples of suppliers of RNA isolation kits are Ambion (Woodward, Tex.), BD-ClonTech (Palo Alto, Calif.), Invitrogen (Carlsbad, Calif.), Sigma Chemical Co. (St. Louis, Mo.), Qiagen (Valencia, Calif.), Promega Inc. (Madison, Wis. etc. The present invention further provides for the utilization of specialized kits which facilitate isolation of RNA from limiting sources of tissue sample or tissues with high RNase content e.g. RNeasy Fibrous Tissue Kit, RNeasy Lipid Tissue Kit etc. (Qiagen, Valencia Calif.). In a particular embodiment, RNA may be isolated from of a tissue sample utilizing Laser Capture Microdissection to select a small population of specific cells (Emmert-Buck et al. 1996, Science 274(5289): 998-1001) followed by isolation of RNA. In another embodiment total RNA from fresh-frozen or formalin-fixed paraffin-embedded tissue may be isolated using appropriate non-commercial procedures or commercially available kits such as High Pure™ RNA Paraffin Kit, (Roche Molecular Systems, Alameda, Calif.), Optimum™ FFPE RNA Isolation Kit, Ambion (Woodward, Tex.) etc. In a further embodiment, the test or standard RNA isolated and purified by the methods described above may be additionally selected for polyadenylated mRNA species by selecting poly-A⁺ RNA utilizing an appropriate format of oligo-dT including but not limited to an oligo-dT cellulose matrix, oligo-dT beads or oligo-dT coated magnetic matrices.

### 5.3 Step (ii) Synthesis of cDNA

The present invention provides for a method to synthesize cDNA from an isolated RNA preparation, which may be a test RNA or a standard RNA. cDNA synthesis may be performed on total RNA or oligo dT selected poly-A⁺ RNA. The invention provides for a primer that may be of any appropriate length so as to generate a specific cDNA by priming a defined region at the 3′-end of mRNA purified from a teat or standard RNA sample. As provided for by the instant invention, cDNA synthesis may be performed utilizing a generic commercially available or custom synthesized oligo dT primer including but not limited to primers from 15 to 26-nucleotides in length. In one embodiment, an oligo-dT primer may or may not be “anchored” at the 3′-end by a G-, C- or T-residue. In another embodiment, the primer may in addition be a mixture of anchored primers comprising of equal proportions of G-, C- or T-residues at the 3′-end of an oligo-dT sequence.

The present invention most preferably contemplates priming cDNA synthesis from within the 3′ poly-A tail or at the junction between the poly-A tail and remaining mRNA sequence that is 5′ to the tail. In a particular non-limiting embodiment, a gene-specific antisense primer located at the 3′-end of a test or standard RNA may also be used to generate a cDNA. When utilized in this manner, only a specific target RNA corresponding to a gene specified by the primer will be reverse transcribed; as opposed to synthesis of a population of cDNAs. This variation does not preclude the utilization of the invention as contemplated. The resulting “test” or “standard” cDNA may then be analyzed as encompassed in the instant invention to quantitate the relative amount of 5′-end and thereby the quality of the test RNA sample of unknown quality from which it was originally isolated.

In non-limiting embodiments, cDNA synthesis may be performed utilizing non-commercial or commercially available cDNA synthesis kits. Commercially available cDNA synthesis kits include but are not limited to the Transcriptor™ First Strand cDNA Synthesis Kit (Roche Molecular Systems, Inc., Alameda, Calif.), SMART™ PCR cDNA Synthesis Kit (BD-Clontech, Palo Alto, Calif.), Superscript™ First-Strand Synthesis System or SuperScript™ III Platinum® (Invitrogen Inc., Carlsbad, Calif.) etc. The invention provides for cDNA synthesis to be performed using any type of enzyme known to the art including native or “enhanced” reverse transcriptases derived from retroviruses such as AMV (Avian Myeloblastosis Virus), or MoMuLV (Moloney Murine Leukemia Virus). The invention encompasses variations of cDNA synthesis procedures including but not limited to use of thermostable polymerases possessing reverse transcriptase activity e.g. MasterAmp™ Tth DNA Polymerase, derived from the thermophilic bacterium Thermus thermophilus, a thermostable enzyme that has both DNA-directed DNA polymerase activity and efficient RNA-directed reverse transcriptase activity. An additional non-limiting variation in the reverse transcriptase reaction may include additives which reduce secondary structure of RNA, enhance enzyme stability and/or enhance the processivity of a reverse transcriptase including but not limited to addition of betaine or other commercially available formulations such MasterAmp™ PCR Enhancer (Epicenter, Madison, Wis.).

Therefore as set forth in the present invention, the determination of quality of a test RNA may be based on many flexible parameters, contingent on the needs of a specific situation. The invention provides for selection of one or several target RNAs, standard RNAs, reverse transcriptases, cDNA synthesis reagents, reaction conditions etc., as set forth in paragraphs above.

### 5.4 Step (iii) Quantitative or Real Time PCR to Quantitate 5′-End and 3′-End Specific Regions

### 5.4.1 PCR Systems

The present invention provides for the quantitation of relative amounts of 5′-end specific and 3′-end specific regions of a selected target mRNA by performing Quantitative or Real Time PCR (Higuchi et al., 1993, Biotechnology 11(9):1026-30; Foley et al., 1993, Trends Genet. 9(11):380-5; Heid et al., 1996, Genome Res. 6(10):986-94). The present invention further provides for a choice of several types of Real Time PCR systems including alternative amplification/labeling protocols, single probe versus multiple probe use, one- versus two- or multicolor based detection and corresponding detection systems, alternative Thermostable polymerases, alternative analytical software, alternative probes including but not limited to LightCycler probes 15 to 40 mers (optimal length: 20 to 30 mers), Dual-labeled fluorogenic probes: 15 to 40 mers, Molecular Beacons: 15 to 40 mers, Scorpions probes: 30 to 60 mers (uni-probe) 15 to 45 mers (bi-probe) etc. The present invention provides for use of commercially available Real Time PCR systems and associated custom reagents, PCR reaction tubes, capillaries, plates, reaction additives and enhancers etc. including but not limited to systems such as the LightCycler® System Family (Roche Molecular Systems, Alameda, Calif.), 7900HT Fast Real-Time PCR System (Applied Biosystems, Foster City, Calif.), Smart Cycler II® thermal cycler (Cepheid, Sunnyvale, Calif.), iCycler (BioRad Inc. Hercules, Calif.) etc. The invention provides for utilization of the two major types of detection and quantitation systems presently in use in Real Time PCR. One system is based on Fluorescence Resonance Energy Transfer (FRET) and includes the TaqMan® Probe, Molecular Beacon and Scorpion primer systems. The other detection and quantitation system is based on SYBR Green chemistry. The working example (Section 6) is based on SYBR Green detection and is equally amenable to a FRET Probe based systems with suitable TaqMan®, Molecular Beacon and Scorpion probes. The present invention additionally provides for but is not limited to new or alternative PCR product quantitation chemistries, detection systems, enhanced software etc. including variations of the TaqMan®, Molecular Beacon, Scorpion and SYBR Green systems. In one embodiment, the Real Time PCR quantitation of 5′-end specific and 3′-end specific regions of several selected target mRNAs may be performed simultaneously in a single reaction i.e. multiplexing with separate monitoring and quantitation of each product or alternatively quantitation may be performed in separate reactions.

### 5.4.2 Choice of PCR Primers

The invention encompasses the selection and utilization of PCR primers which amplify 5′-end specific and 3′-end specific regions of the selected target mRNA, converted to target cDNA by the reverse transcription cDNA synthesis step described above. The invention provides for sense and antisense PCR primers of any nucleotide length so as to permit specific Real Time PCR amplification of 5′- and 3′-specific regions of the selected target mRNA. More preferably oligonucleotide primers for Real Time PCR may range from 15-30 nucleotides depending on the parameters known to one skilled in the art used to design a primer for PCR reactions.

The invention therefore provides for primer design based on considerations such as melting temperature, secondary structure, G-C content etc. Most preferably the oligonucleotide primers used to amplify the 5′- and 3′-regions of the target cDNA may be between 18 and 25 nucleotides.

In one embodiment, the specific site for annealing in the CDNA (i.e. the exact sequence of a PCR primer) may be determined by parameters routinely used in primer design as described above to ensure specificity of primer binding and robust PCR. Further, the invention provides for the sequence of primer pairs for amplification of the 5′-region of the target mRNA as disclosed in the invention to be within any region spanning the first nucleotide at or around the 5′-terminus of the mature mRNA and extending to a region encompassing preferably up to 30% of the length of the full mRNA in the downstream or 3′ direction. More preferably, the invention provides for primers to be chosen from a region at or around the 5′-terminus of the target mRNA and encompassing 20% or 15% or most preferably 10% of the length of the full mRNA downstream from the 5′-terminus. The primer pair chosen to amplify the 5′-end of the target mRNA is not restricted to the 5′-extremity and may initiate at a defined sequence downstream of the 5′-end of the target RNA. The invention also provides for an antisense primer for the 5′-end that may be from any region so as to be spaced apart from the sense primer to generate a PCR product based on the range limitations described i.e. within 30% of the 5′ end of the mRNA sequence.

The invention further encompasses any length of amplified PCR product so as to be compatible with a range of primer annealing sites, preferably within 30% of the 5′ end of the mRNA sequence. More preferably, the range of PCR product size may be from between 50 to 500 nucleotides. Most preferably, the range of amplified product size may be from 150-300 nucleotides.

In another embodiment, the sense and antisense primer sequence for amplification of the 3′-region of the target mRNA as disclosed in the invention may be within any region spanning from a site within the poly-A tail retained after cDNA synthesis capable of hybridizing to an oligo-dT primer and extending to a region encompassing preferably up to 30% of the length of the full mRNA in upstream or 5′ direction from the last residue of the poly-A terminus. In another embodiment the antisense primer for PCR amplification of the 3′-end of the target mRNA may start at the junction between non-polyA sequence and the polyA tail and extend upstream. In a further embodiment, the sense and antisense primer pairs may be chosen in any region at the 3′-end of the target rnRNA so as to encompass a region spanning the equivalent of up to 30% of the total length of the target mRNA and starting at the 3′-terminus. More preferably the choice of primer may be within a region encompassing up to 25%, 20% or 15% of the full length of the target mRNA immediately 5′ to the poly-A tail. Most preferably, the choice of primers may be made from a region encompassing 10% of the 3′-terminal region of the target mRNA.

In one embodiment, the primers used in PCR reactions to amplify the 5′-end specific and 3′-end specific regions of the selected target mRNA may be further comprised partially or entirely of unmodified bases or of modified bases as known in the art including but not limited to phosphorothioate modification. In another embodiment, the primers used in PCR reactions to amplify the 5′-end specific and 3′-end specific regions of the selected target mRNA may contain mismatched nucleotides to the target sequence or be modified by addition of side chains containing but not limited to a biotin or a fluorescent tag.

### 5.5 Step (iv): Calculating the Relative Abundance of

### 5′-End in a Test or Standard RNA

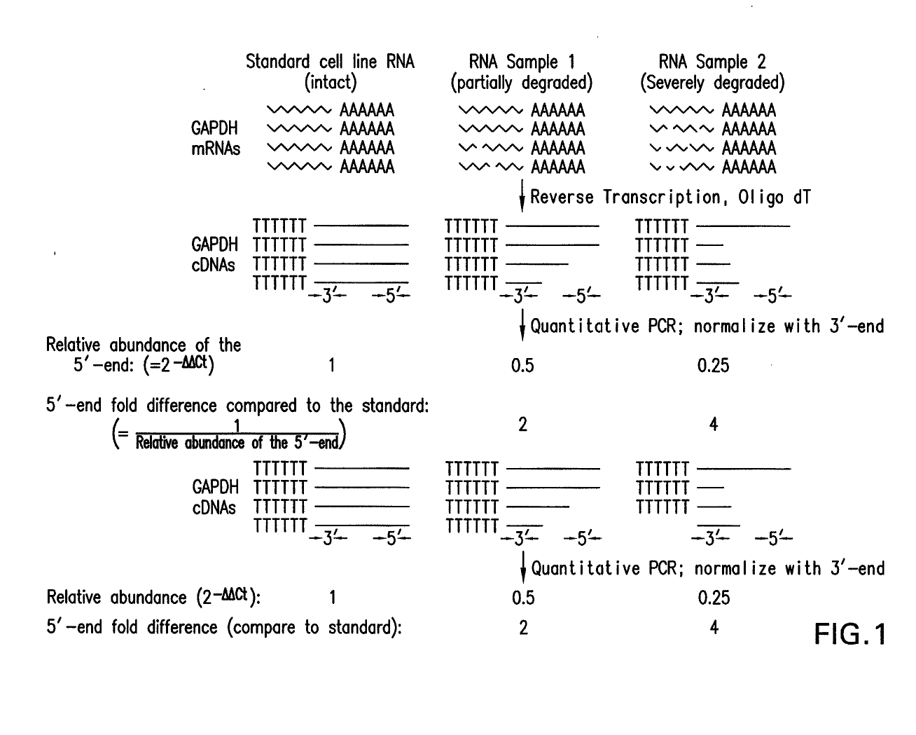

The present invention encompasses a method for calculating the relative amount of 5′-end containing RNA species in a sample. The relative amount of 5′-end containing RNA species of a test RNA reflects the overall quality of that sample. Normalization of the amount of 5′-end in a given sample is based on the assumption and experimentally determined finding that the ratio of 5′-end specific mRNA sequence to 3′-end specific mRNA sequence is equal to or tends toward a value of 1 when the mRNA is of good quality (FIG. 1) and paragraphs below. Thus as a first step the relative amount of 5′-end of a target mRNA is determined by normalizing its amount to that of the amount of 3′-end of the same mRNA in a given test RNA. Thus, when a test RNA is normalized to a standard RNA using the relative abundance of the 5′-end of the target RNA species, the value obtained will be correlated to both the 3′-end to 5′-end ratio, obtained utilizing the GeneChip® Test3 array and to the quality of the test RNA.

A test RNA is usually degraded or hydrolyzed to a greater extent at the 5′ end before complete degradation. When cDNA synthesis is performed using primer binding to a 3′ location such as oligo-dT, a degraded test RNA will generate a higher proportion of partial 3′-end containing cDNA molecules compared to full length molecules containing both 3′- and 5′-ends (FIG. 1). Thus the normalized relative abundance of a 5′-end of target mRNA will range from a value of 1 if undegraded, to smaller fractions of 1 depending on the extent of degradation. The present invention provides for a method to express the quality of an isolated sample of RNA and assign a relative numerical value or rating to the RNA quality of a sample for comparison with other samples. Quantitation of the amount of 5′-end of a target mRNA in a test RNA or standard RNA and the amount of 3′-end of a target mRNA in a test RNA or standard RNA is determined by quantitative Real Time PCR as described above and the working example in Section 6.

In particular, the invention provides for a method wherein the quality of a RNA preparation is obtained by determining the relative amount of a target by the Delta-delta method (Livak and Schmittgen, 2001, Methods 25:402-408) using the formula: Relative Abundance =2^{-ΔΔCt}, where (i) ΔΔCt=[(Ct5′ test −Ct3′ test)−(Ct5′ standard−Ct3′ standard)]; and (ii) Ct is Threshold Cycle, which reflects the cycle number at which the fluorescence generated within a reaction crosses the “threshold” which is the point during the PCR reaction at which a sufficient number of amplicons have accumulated, in that sample, to be at a statistically significant point above the baseline. The invention provides for the threshold to be set at a point of exponential amplification in the PCR reaction, as opposed to the plateau phase or in the initial linear phase of amplification. Setting the threshold in the exponential phase as provided by the invention, ensures that measurement of PCR products is quantitative. As seen in FIG. 1, the relative abundance of the 5′-end of a target mRNA in the standard RNA is arbitrarily set at 1 as the reference point to compare with the test RNA. The standard RNA is of good quality when the amount of 5′ and 3′, amplicons derived from the target mRNA is in equal amount. The invention provides for the 2^{-ΔΔCt} value of a test RNA to tend toward a smaller fractional value as the sample is more degraded.

### 5.6 Step (v) Determining Quality of a Test RNA by Calculating the 5′-End Ratio

The invention provides for a comparative evaluation of the quality of a set of test RNAs of unknown quality to be expressed as a “5′-end fold difference” value (i.e. relative abundance of target RNA species in standard RNA/relative abundance of target RNA species in test RNA) wherein a smaller numerical value closer to 1 indicates a better quality test RNA. Thus, the final step of the invention essentially encompasses the method of determining the quality of an RNA preparation by determining the relative amount of 5′-end specific molecule present in a preparation of unknown quality compared to 5′-end specific molecule present in a high quality standard RNA sample by performing prior steps (i) through (iv) of the invention. In non-imiting embodiments of the invention, the 5′-end fold difference is at least 2 and at most 10, preferably between 1.5 and 6 and most preferably between 1 and 3.

### 6. USES OF THE INVENTION

### 6.1 Advantages of the Invention Over Alternate Methods

Traditionally, the quality of a test RNA has been determined by resolving the individual species of a purified RNA on a gel based on molecular weight, usually by denaturing gel electrophoresis. This step is followed by visualizing the separated 18S and 28S ribosomal RNA bands and determining their integrity based on sharpness of the bands as well as whether there is an approximately 2-fold greater intensity of the 28S band. This method may be relatively easy and performable on a routine basis. However, the major purpose of isolating RNA to detect and analyze protein coding messenger RNA molecules are be visualized by the electrophoresis method described above due to heterogeneity in size and low abundance of mRNA (between 1-5% of total cellular RNA). Therefore determining the quality of mRNA by electrophoresis based methods is at best indirect, subjective, and difficult to perform when the test RNA quantity is limiting, lacks in accuracy, and does not afford the possibility of numerically grading multiple test RNAs to denote the relative quality of each. The Agilent Bioanalyzer® is a capillary electrophoresis based system combined with a fluorescence detection system. This instrument may be utilized to determine the quality of a test RNA based on the principle of measuring and quantitating the amounts of 28S and 18S rRNA and determining the ratio of 28S to 18S rRNA species, a value closer to two indicating better quality. While the Bioanalyzer® overcomes certain limitations of the more commonly used electrophoresis based gel visualization system, it suffers from disadvantages including measuring 28S and 18S rRNA rather than mRNA molecules. While analysis in the Bioanalyzer® requires small amounts of test RNA and multiple test RNAs may be analyzed, the cost of this equipment precludes its utilization in many research settings. Thus the Bioanalyzer® when used as a means to determine RNA quality suffers from significant drawbacks. An alternative method to determine the quality of test RNA involves microarray analysis utilizing the GeneChip® Test3 array (Affymetrix, Santa Clara, Calif.). The array is specifically designed to determine the quality and suitability of a test RNA prior to performing full scale microarray analysis. The GeneChip® Test3 array contains probes derived from the 5′, middle and 3′ portions of eukaryotic house keeping genes (Warrington et al., 2000 Physiol Genomics, 2:143-147) in addition to other sequences, thereby permitting the evaluation of RNA quality by determining the extent of RNA degradation based on hybridization of unknown quality test RNA samples to region specific gene probes. Utilization of the GeneChip® Test3 array requires access to GeneChip® istrumentation, consumables including the GeneChip® Test3 array itself and the associated costs related to its usage. Analysis of multiple test RNAs using this system to merely determine RNA quality, before performing the actual analysis could significantly increase costs of performing a study and may in addition have reduced feasibility if the quantity of test RNA is limiting. The present invention provides for a method to evaluate RNA quality based on Real Time PCR methodology which overcomes several of the drawbacks noted in the alternative methods above. The present invention provides for the analysis of RNA quality by measuring the integrity of an actual target mRNA of choice rather than an indirect measure utilizing rRNA. The costs associated with performing the method of the instant invention are likely to be significantly lower than either the Agilent Bioanalyzer® or use of the GeneChip® Test3 array system. The instant invention provides for utilization of Real Time PCR wherein the equipment and consumables costs are significantly lower than either the Bioanalyzer® or GeneChip® systems. The present invention provides for considerable flexibility and the facility to analyze a given set of test RNAs using a variety of target mRNAs (e.g. high and low abundance mRNAs) as needed, to obtain a definitive determination of RNA quality. The present invention uses as little as 5 μg of purified test RNA to perform the analysis as opposed to the μg amounts of RNA required by all the other alternatives. In addition, the present invention provides for the possibility of determining the quality of a relatively large number of test RNAs in a comparatively short time. The present invention thus provides for a rapid, cost-effective and validated means of determining RNA quality compared to existing methods that are variously, less accurate, more complex to perform, require large resource investments in complex equipment, are expensive to perform and require a relatively long time to obtain results.

### 6.2.1 Uses of the Invention for Microarray Analysis Sample Screening

The present invention provides for a method to determine the quality of a test RNA prior to its utilization in a microarray analysis. The instant invention provides an inexpensive and efficient way to pre-screen a test RNA prior to further analyses including but not limited to microarray analysis. In one embodiment, it is possible to eliminate low-quality test RNAs and chose comparable ones for further analyses including but not limited to microarray analysis. The invention further provides for a method to determine the quality of a test RNA before a microarray analysis to detect the presence of differentially expressed genes. The invention in another embodiment provides for a method to determine the quality of a test RNA before a microarray analysis for series of one or more sets of test RNAs to detect the presence of differentially expressed genes. The invention provides a method to survey the quality of test RNAs in a large number of individual samples in an efficient and cost-effective manner. In a particular embodiment, the said series of test RNAs may be part of a survey including but not limited to a survey based on molecular disease diagnosis, a disease prognosis survey, a survey or study for staging the progression of a disease, an integrated disease diagnosis and treatment method, an epidemiological survey, an epidemiological survey particularly directed to detection of one or more disease susceptibility genes, an epidemiological survey particularly directed to the detection of one or more mutated genes, an epidemiological survey particularly directed to the detection of one or more cancer susceptibility genes or an epidemiological survey particularly directed to the detection of one or more factors potentially involved in disease caused due to environmental, industrial or radiological pollution. In a further embodiment the invention may be used to determine the quality of a test RNA to be used in drug discovery. In a further embodiment the invention may be used to determine the quality of a test RNA to be used to determine the cause of an allergy, response to an allergen or response to an anti-allergic treatment. In a further embodiment of the invention, the quality of RNA is determined in a sample or set of samples that contain differentially expressed genes to characterize the differences between two related systems under study. In particular non-limiting examples the related systems may be an untreated cell or tissue being compared to a treated cell or tissue. In another non-limiting example, the present invention may be used for characterization of the gene expression profiles of one or more sets of related tissue biopsies or one or more sets of related post-mortem tissues samples. The present invention provides for a method to determine the quality of a stored test RNA to be used in a microarray or any alternative form of analysis known in the art. In one embodiment, the invention provides for a method to evaluate a test RNA to determine its complexity for presence of full length and rare messages before construction of a cDNA library. In another embodiment, the quality of a cDNA library may be tested to determine the frequency and occurrence of full length cDNA or the extent of complexity of the library by testing for the presence and amount cDNAs corresponding to rare versus more abundant mRNAs.

### 6.2.2 Use of the Invention in a Kit

In still further embodiments, the present invention provides for kits for practicing the methods of the invention. Such kits may contain, for example but not by way of limitation, PCR primers, labeled nucleic acid probes, and/or nucleic-acid bearing chips or blots which may be used to determine the quality of a test RNA according to the present invention.

In non-limiting examples the present invention provides for a kit comprising primer pairs, deoxyribonucleotide (dNTP) mixes, reaction buffer systems, fluorescent detection reagents for Real Time PCR, purified standard RNAs, positive and negative control cDNAs, Reverse Transcriptases and Thermostable polymerases. In one embodiment, the reagents provided in kit form are packaged in pre-calibrated amounts to perform a specific number of tests to determine the quality of a test RNA. In another embodiment, the primers provided in the kit may comprise a minimum set of four primers, one pair each to quantitate the 5′-end and the other pair to quantitate the 3′-end of a target mRNA. In another embodiment, the kit provides for preferably at least four, at least five, at least six, at least seven, at least eight, at least nine or at least ten such 5′-end and 3′-end primer pairs which enable quantitation of several distinct target mRNAs to thereby evaluate the quality of a test RNA. In a non-limiting example the target RNAs may be drawn from a group comprising of but is not limited to preferably at least one, at least two, at least three, at least four, at least five, at least six, at least seven, at least eight, or at least nine of the following genes GAPDH, myc, fos, β-actin, microglobulin, EF-1a, ubiquitin cyclophilin or rRNA. In an embodiment, the kit provides for positive control target cDNAs corresponding but not limited to the primers provided in the kit. In another embodiment, the kit provides for purified positive control target mRNA corresponding but not limited to the primers provided in the kit. In a non-limiting embodiment the positive control target cDNA or mRNA may be drawn from but is not limited to a group selected from preferably at least one, at least two, at least three, at least four, at least five, at least six, at least seven, at least eight, or at least nine of the following genes: GAPDH, myc, fos, β-actin, microglobulin, EF-1a, ubiquitin, cyclophilin or rRNA gene sequences. In another embodiment, the invention provides for a reverse transcriptase enzyme to synthesize a cDNA according to the invention. In another embodiment the reverse transcriptase is selected but not limited to a group of enzymes including native unmodified reverse transcriptases, modified reverse transcriptases, an AMV enzyme, a MoMuLV enzyme, a derivative of an AMV or a MoMuLV enzyme, a thermostable enzyme possessing reverse transcriptase activity etc. In another embodiment, the invention provides for a thermostable polymerase that may be a mixture of more than one enzyme so as to in combination provide both fidelity and high processivity. In another embodiment, the invention provides for fluorescent detection reagents including but not limited to a SYBR green reagent, a TaqMan® Probe system or any alternative detection system known in the art to detect and quantitate Real Time PCR products.

### 7. WORKING EXAMPLE

### 7.1 Materials and Methods

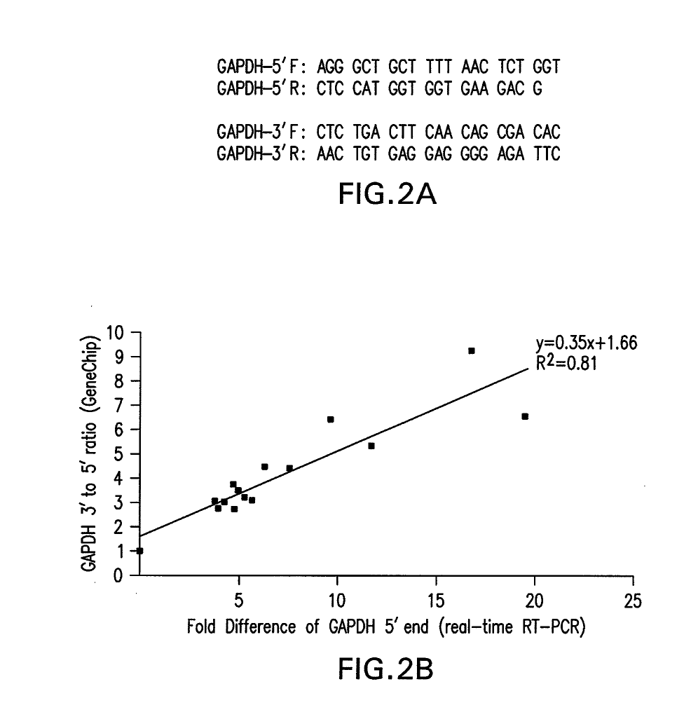

RNA isolation. cDNA synthesis and Real-time PCR analysis. For each sample, 5 μg of purified total RNA was used to generate cDNAs with the Superscript™ First-Strand Synthesis System (Invitrogen, Carlsbad, Calif.). The final reaction volume was 20 μl. Two sets of PCR primers (FIG. 2A) that flanked the oligomer sequences of the 5′ and 3′ GAPDH probes on the Test3 array (Affymetrix, Santa Clara, Calif.) were designed. PCR generated 262 bp and 276 bp products for the 5′ and 3′ ends of GAPDH respectively. PCRs were performed using the Brilliant SYBR Green fluorescence dye (Invitrogen, Carlsbad, Calif.), the OmniMix™ HS PCR beads (TaKaRa Bio Inc, Otsu, Shiga, Japan), and the Smart Cycler II thermal cycler (Cepheid, Sunnyvale, Calif.). Each 25 μl PCR reaction contained PCR primers (1 pM for each primer), SYBR Green (1×), OmniMix™ PCR reagent (one bead for 2 reactions) and 0.5 μl of cDNA. The following protocol was employed: 1 cycle of 5 min at 95° C. and then 40 cycles of 15 s at 95° C., 30s at 60° C., 30s at 72° C. and 20s at the temperature that fluorescence intensity was measured (87° C. for 5′-end PCR and 85° C. for 3′-end PCR).

Microarray analysis. The Genechip® Test3 array (Affymetrix, Santa Clara, Calif.) was used to evaluate the quality of RNA preparations by microarray analysis. Total RNA was isolated from brain samples by Trizol® reagent (Invitrogen, Carlsbad, Calif.) and was used to prepare biotin-labeled complementary target RNA for hybridization according to manufacturer's protocols. RNA samples that produce a ratio of 3′ end to 5′ end of GAPDH transcript with a value less than or equal to 4 on the Genechip® Test3 array (Aymetrix, Santa Clara, Calif.) was routinely obtained using the procedure described above.

### 7.2 Results and Discussion

The method takes advantage of the fact that when oligo-dT is used for priming in a reverse transcription reaction, a better quality test or standard RNA generates more full-length cDNAs and a partially degraded test or standard RNA gives rise to fragmented short cDNAs that might or might not contain the 5′ portion of the original messages. Hence, if two synthesized cDNA pools are normalized by comparing the 3′ end of a house keeping gene such as GAPDH, the relative abundance of the 5′ end of GAPDH in the pool correlates with the relative quality of the original RNA (FIG. 1). RNA isolated from cultured cell lines grown in vitro are of high quality and produce a value close or equal to 1 for the 3′- to 5′-end ratio of GAPDH on a Test3 array (Affymetrix, Santa Clara, Calif.). To obtain a comparative measure utilizing quantitative RT-PCR, the relative fold difference of the 5′ end of GAPDH transcripts between 14 human brain samples and a standard RNA that was isolated from SW 13 cells, a human adrenal carcinoma cell line was performed. The data was plotted onto a graph to study the relationship between the two methods, quantitative PCR and the Genechip® (Affymetrix, Santa Clara, Calif.) method for monitoring RNA quality.

Normalized by the 3′-end, the relative abundance of the GAPDH 5′-end in each test RNA as compared to that of the cell line derived standard RNA was calculated by the Delta-delta method (Livak and Schmittgen, 2001, Methods 25:402-408): amount of target or relative abundance 2^{-ΔΔCt}, where ΔΔCt=[(Ct5′ test−Ct3′ test)−(Ct5′ standard −Ct3′ standard)]; Ct=threshold cycle. The relative amounts of the GAPDH 5′-end in the test RNAs were always smaller than in the standard RNA derived from a cell line. Therefore, fold difference =1/(relative abundance). Each data point in FIG. 2B represents the value of a single brain specimen. A total of 14 specimens were tested. The data point at coordinate (0, 1) is from a human cell line that showed no degradation. Each data point (FIG. 2B) is the average of three separate reactions. In a typical reaction, the 3′-end Ct value fell between 18-20 cycles. Although the 3′-end Ct value gave a crude indication of the quality of a test RNA, it was found that no direct relationship to the integrity of the GAPDH transcripts as revealed by Test3 array was obtained by relying on 3′-end Ct value determinations. In contrast, as illustrated in FIG. 2B, the relative fold difference of 5′ end obtained by the real-time RT-PCR based method exhibited a linear correlation R²=0.81) with the 3′- to 5′-end ratio of GAPDH generated by Test3 array (Affymetrix, Santa Clara, Calif.). Better-quality test RNA yielded a GAPDH 3′- to 5′-end ratio of <4 by Genechip® and displayed a<6 fold difference of the 5′-end as compared to that of the standard RNA from a cell line, by real-time RT-PCR.

In the experiments described in FIG. 2B, RNA extracted from SW13 cells was utilized as the standard RNA. However, RNA obtained from other human cell lines may also be used for this purpose. In addition, this method can also be applied to analyze the integrity of other transcripts including those from genes known to be relatively unstable such as c-myc or c-fose.

Various publications, including journal articles, electronic documents, patents and patent applications are cited herein, the contents of which are hereby incorporated by reference in their entireties.

I claim:

1. A method for evaluating RNA quality from a biological sample comprising:
(i) determining the relative normalized amount of 5′-end of a target mRNA in a test RNA of unknown quality and a standard RNA of known, good quality;
(ii) determining the numerical ratio of 5′-end abundance in standard to test RNA; and
(iii) rating the relative quality of one or more unknown test RNAs based on the numerical value obtained for each sample of test RNA where a smaller ratio indicates that the RNA is of better quality.

2. The method of claim 1 wherein the test or standard RNA is selected from a group consisting of total RNA and poly adenylated RNA.

3. The method of claim 1 wherein cDNA is synthesized from the test or standard RNA utilizing a primer hybridizing to the polyA tail of the test or the standard RNA.

4. The method of claim 1 wherein quantitation of a target mRNA in the test RNA and the standard RNA is made by performing quantitative or real time PCR on cDNA derived from the test and standard RNA using a primer pair to quantitate a region in the 5′-end of a target mRNA, and a primer pair to quantitate a region in the 3′-end of the same target mRNA.

5. The method of claim 1 wherein calculating the relative abundance of the 5′-end of the target mRNA in the test RNA population is performed using the 2^{-ΔΔCt} method.

6. The method of claim 1 wherein the quality of the test RNA is determined by calculating the 5′-end fold difference of a target mRNA in the test RNA when compared to the standard RNA.

7. The method of claim 1 wherein the source of test RNA and standard RNA is a non-malignant cell or tissue selected from a group consisting of brain cells, spinal cord cells, skin cells, throat cells, tongue cells, lung cells, stomach cells, intestine cells, liver cells, pancreas cells, kidney cells, breast cells, ovary cells, testicle cells, prostate cells, bone cells, muscle cells, lymphocyte cells, erythroblast cells, macrophages and dendritic cells.

8. The method of claim 1 wherein the source of test and standard RNA is a malignant cell or tissue selected from a group consisting of a glioblastoma cell, an cancer cell, a pancreatic cancer cell, a renal cancer cell, a breast cancer cell, an ovarian cancer cell, a prostate cancer cell, a bone cancer cell, a connective tissue cancer cell, a leukemia cell, a lymphoma cell, and a melanoma cell.

9. The method of claim 7 or 8 wherein the quality of the test RNA is determined in a sample derived from tissue that has been subjected to laser capture or other microdissection procedures to obtain specialized cell types.

10. The method of claim 7 or 8 wherein the quality of the test RNA is determined in a sample derived from a group consisting of a fresh tissue sample, a frozen tissue sample, a fixed tissue section sample, a cryostat section sample, paraffin embedded section sample and a tumor bank sample and a tissue bank sample.

11. The method of claims 1, 7 or 8, to perform analysis of multiple test RNA samples to determine RNA quality prior to microarray analysis of said test RNA.

12. A kit for practicing the method of claims 1, 7, or 8, to perform analysis of multiple test RNA samples to determine RNA quality prior to microarray analysis of said test RNA.

13. The method of claim 9 to perform analysis of multiple test RNA samples to determine RNA quality prior to microarray analysis of said test RNA.

14. The method of claim 10 to perform analysis of multiple test RNA samples to determine RNA quality prior to microarray analysis of said test RNA.


SEQUENCE LISTING (4 sequences)

SEQ ID NO: 1 - DNA, 21 bp, Artificial Sequence
  synthetic oligonucleotide
agggctgcttttaactctggt

SEQ ID NO: 2 - DNA, 19 bp, Artificial Sequence
  synthetic oligonucleotide
ctccatggtggtgaagacg

SEQ ID NO: 3 - DNA, 21 bp, Artificial Sequence
  synthetic oligonucleotide
ctctgacttcaacagcgacac

SEQ ID NO: 4 - DNA, 21 bp, Artificial Sequence
  synthetic oligonucleotide
aactgtgaggaggggagattc